(19) United States
(12) Patent Application Publication
(10) Pub. No.: US 2020/0345407 A1
(43) Pub. Date: Nov. 5, 2020

(54) GENERATOR AND OPERATION METHOD THEREOF

(71) Applicant: OLYMPUS CORPORATION, Tokyo (JP)

(72) Inventors: Kizuku INABA, Hachioji-shi (JP); Kenichi KIMURA, Hachioji-shi (JP); Satoshi HONDA, Hachioji-shi (JP)

(73) Assignee: OLYMPUS CORPORATION, Tokyo (JP)

(21) Appl. No.: 16/936,088

(22) Filed: Jul. 22, 2020

Related U.S. Application Data
(63) Continuation of application No. PCT/JP2018/001974, filed on Jan. 23, 2018.

(51) Int. Cl. A61B 18/12; A61B 18/14

(52) U.S. Cl. CPC A61B 18/1206; A61B 2018/0019; A61B 18/14

(57) ABSTRACT

A generator that includes a drive circuit connected to a surgical instrument including a vibration system having a probe and an ultrasonic transducer, which supplies power to the ultrasonic transducer. The control circuit detects a first trigger causing the control circuit to perform a resonance-point search on the vibration system. Upon detecting the first trigger, the control circuit determines a resonance point of a frequency of vibration of the probe by performing the resonance-point search, and upon determining the resonance point, causes the drive circuit to execute a standby operation. The control circuit then detects a second trigger causing the surgical instrument to treat the living tissue, and upon detecting the second trigger, causes the drive circuit to execute an actual output operation by increasing the supply of power to the surgical instrument such that the surgical instrument treats the living tissue.

### CROSS-REFERENCE TO RELATED APPLICATIONS

This application is a Continuation Application of PCT Application No. PCT/JP2018/001974, filed Jan. 23, 2018, the entire contents of which are incorporated herein by reference.

### FIELD

Embodiments described herein relate to a generator and an operation method of the generator.

### BACKGROUND

A known electric scalpel is configured to coagulate and cut a living tissue as a treatment target by applying a high-frequency current thereto during surgery. To prevent a living tissue from sticking to a scalpel edge, a technique of vibrating an electrode, which is the scalpel edge, at a frequency in an ultrasonic region is known in such an electric scalpel. For example, whether a scalpel edge and a living tissue are in contact with each other is determined based on an electric impedance which is measured by applying a high-frequency current. When the scalpel edge and a living tissue are determined to be in contact with each other, an output of a high-frequency current and an output of vibration are set to preset values for treatment.

### SUMMARY

According to a first aspect, a generator is provided that includes a drive circuit connected to a surgical instrument including a vibration system having a probe and an ultrasonic transducer, which supplies power to the ultrasonic transducer. The at least one control circuit detects a first trigger causing the at least one control circuit to perform a resonance-point search on the vibration system, based on the probe of the surgical instrument contacting a living tissue. Upon detecting the first trigger, determining a resonance point of a frequency of vibration of the probe by performing the resonance-point search on the vibration system, and upon determining the resonance point, causing the drive circuit to execute a standby operation by supplying power to the ultrasonic transducer so as to maintain resonance of the frequency based on the determined resonance point. The control circuit detects a second trigger causing the surgical instrument to treat the living tissue, and upon detecting the second trigger, causing the drive circuit to execute an actual output operation by increasing the supply of power to the surgical instrument such that the surgical instrument treats the living tissue.

According to a second aspect, a method is provided that supplying, by a drive circuit of the generator, a power to an ultrasonic transducer, the drive circuit being connected to a surgical instrument including a vibration system having a probe and the ultrasonic transducer; detecting, by at least one control circuit, a first trigger that is generated in response to the probe contacting a living tissue, the first trigger causing the at least one control circuit to perform a resonance-point search on the vibration system; upon detecting the first trigger, determining, by the at least one control circuit, a resonance point of a frequency of vibration of the probe by performing the resonance-point search on the vibration system; upon determining the resonance point, causing, by the at least one control circuit, the drive circuit to execute a standby operation by supplying power to the ultrasonic transducer so as to maintain resonance of the frequency based on the determined resonance point; detecting, by the at least one control circuit, a second trigger causing the surgical instrument to treat the living tissue; and upon detecting the second trigger, causing, by the at least one control circuit, the drive circuit to execute an actual output operation by increasing the supply of power to the surgical instrument such that the surgical instrument treats the living tissue.

Advantages of the embodiments will be set forth in the description which follows, and in part will be obvious from the description, or may be learned. The advantages may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate embodiments, and together with the general description given above and the detailed description of the embodiments given below, serve to explain the principles.

FIG. 1 is a schematic view showing an exemplary appearance of a surgical system according to one embodiment.

FIG. 2 is a schematic block diagram showing an exemplary configuration of the surgical system according to the embodiment.

FIG. 3 is a schematic flowchart showing an exemplary operation of the surgical system according to the embodiment.

FIG. 4 is a schematic flowchart showing an exemplary operation in a first mode of the surgical system according to the embodiment.

FIG. 5 is a timing chart for illustrating an operation in the first mode of the surgical system according to the embodiment.

FIG. 6 is a timing chart for illustrating an operation in a second mode of the surgical system according to the embodiment.

FIG. 7 is a schematic flowchart showing an exemplary operation in the second mode of the surgical system according to the embodiment.

FIG. 8 is a schematic view showing an exemplary configuration of a surgical instrument including a pressure sensor.

FIG. 9 is a schematic view showing an exemplary configuration of a surgical instrument including an optical distance sensor.

### DETAILED DESCRIPTION

### [Configuration of Surgical System]

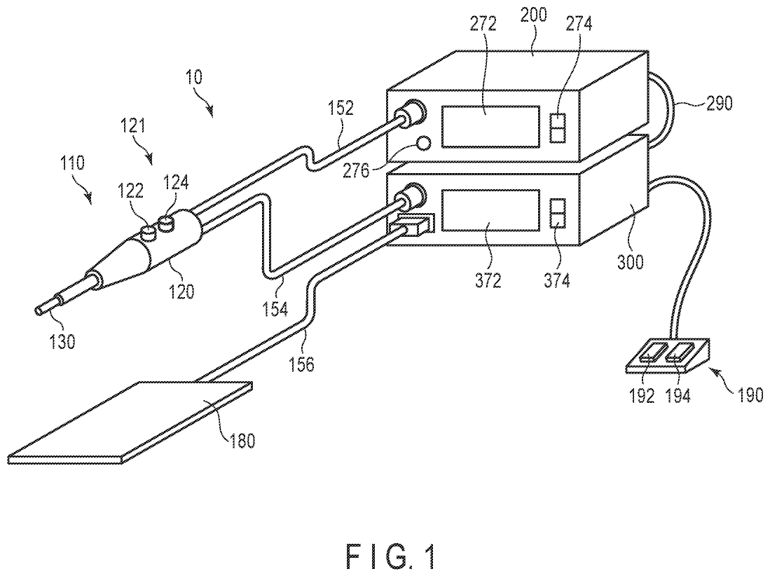

Embodiment(s) will be described with reference to the drawings. FIG. 1 is a schematic view showing an appearance of a surgical system 10 according to the present embodiment. The surgical system 10 is a system for performing treatment on a living tissue as a treatment target using a high-frequency current and ultrasonic vibration. The surgical system 10 operates as a monopolar high-frequency treatment instrument. In the surgical system 10, a probe as an electrode vibrates at a frequency in an ultrasonic region. Vibration of the probe is also utilized for treatment.

As shown in FIG. 1, the surgical system 10 includes a surgical instrument 110, a return electrode 180, a footswitch 190, a first generator 200, and a second generator 300.

The surgical instrument 110 includes an operation unit 120 and a probe 130. The operation unit 120 is a portion (e.g., handle) to be grasped by a user to operate the surgical instrument 110. The probe 130 is provided in a distal end of the operation unit 120. In treatment, the probe 130 is brought into contact with a living tissue as a treatment target. The probe 130 operates as a high-frequency electrode. In treatment, a high-frequency current flows from the probe 130 to a living tissue. The probe 130 is connected to an ultrasonic transducer provided inside the operation unit 120. In treatment, the probe 130 vibrates at a frequency in an ultrasonic region.

The operation unit 120 is provided with an output switch 121. The output switch 121 includes a first switch 122 and a second switch 124. The first switch 122 is a switch relating to an input for the surgical instrument 110 to operate in a cutting mode. The cutting mode is a mode for burning off a portion of a living tissue as a treatment target which comes into contact with the probe 130, by outputting a relatively large high-frequency current. The second switch 124 is a switch relating to an input for the surgical instrument 110 to operate in a hemostasis mode. The hemostasis mode outputs a high-frequency current smaller than that of the cutting mode. In the hemostasis mode, the probe 130 burns off a contact portion of a living tissue as a treatment portion while performing hemostasis treatment by biologically denaturing an edge face of the burnt portion of the living tissue.

The return electrode 180 is adhered to a body surface of a treatment subject. A current output from the probe 130 of the surgical instrument 110 is collected by the return electrode 180.

The footswitch 190 includes a first switch 192 and a second switch 194. The first switch 192 of the footswitch 190 has a similar function to that of the first switch 122 provided in the surgical instrument 110. The second switch 194 of the footswitch 190 has a similar function to that of the second switch 124 provided in the surgical instrument 110. Namely, a user can switch on and off an operation of the surgical instrument 110 by using the first switch 122 and the second switch 124 both provided in the surgical instrument 110, and also by using the first switch 192 and the second switch 194 both provided in the footswitch 190.

The first generator 200 controls an operation of the surgical instrument 110. The first generator 200 outputs a power for driving the ultrasonic transducer of the surgical instrument 110. The first generator 200 is connected to the surgical instrument 110 via a cable 152. The first generator 200 outputs a power to the surgical instrument 110 via the cable 152. The first generator 200 is connected to the second generator 300 via a cable 290. The first generator 200 and the second generator 300 exchange necessary information via the cable 290.

The first generator 200 includes a touch screen 272, an input unit 274, and a microphone 276 each serving as a user interface. The touch screen 272 includes, for example, a liquid crystal display (LCD) and a touch panel. The input unit 274 includes a button switch, etc. The first generator 200 is configured to obtain a user's instruction via the touch screen 272, the input unit 274, and the microphone 276. The first generator 200 is configured to present information to a user via the touch screen 272.

The second generator 300 controls an operation of the surgical instrument 110. The second generator 300 outputs a power in such a manner that a current flows between the probe 130 of the surgical instrument 110 and the return electrode 180. The second generator 300 is connected to the surgical instrument 110 via a cable 154. The second generator 300 is connected to the return electrode 180 via a cable 156. The second generator 300 includes a touch screen 372 and an input unit 374 each serving as a user interface. The touch screen 372 includes, for example, a liquid crystal display (LCD) and a touch panel. The input unit 374 includes a button switch, etc. The second generator 300 is configured to obtain a user's instruction via the touch screen 372 and the input unit 374. The second generator 300 is configured to present information to a user via the touch screen 372.

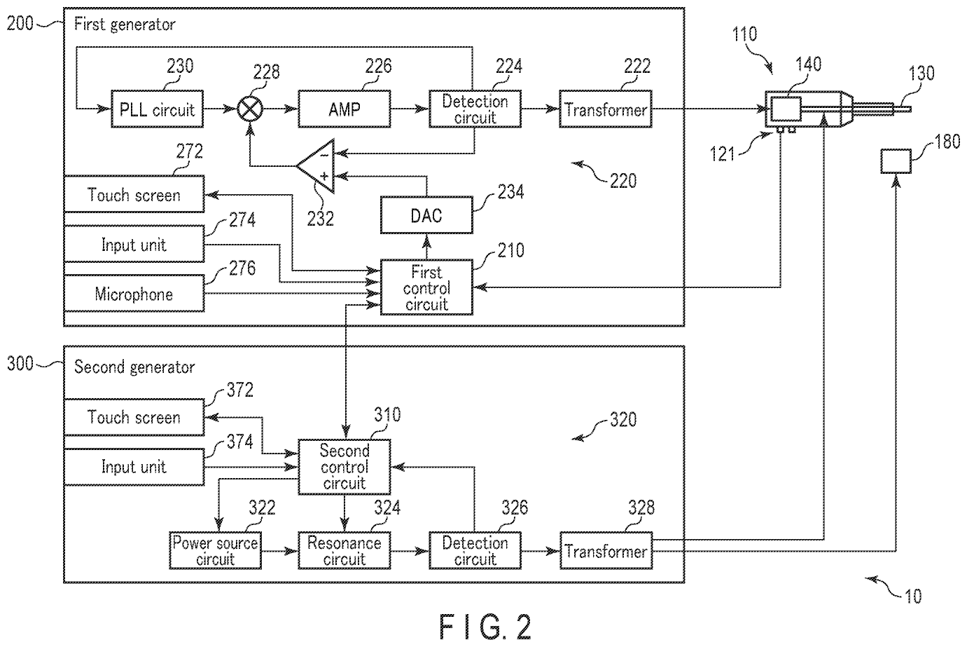

FIG. 2 is a schematic block diagram showing an exemplary configuration of the surgical system 10. As shown in FIG. 2, the surgical instrument 110 is provided with an ultrasonic transducer 140. The ultrasonic transducer 140 includes a plurality of piezoelectric elements. Each of the piezoelectric elements is sandwiched between electrodes mutually different in polarity. These piezoelectric elements are stacked to form the ultrasonic transducer 140. Upon receipt of AC voltage of a frequency in an ultrasonic region applied from the first generator 200, the ultrasonic transducer 140 vibrates at this frequency. This vibration is transmitted to the probe 130.

The first generator 200 includes a first control circuit 210. The first control circuit 210 controls an operation of each element in the first generator 200. The first control circuit 210 is formed by an integrated circuit such as, e.g., a Central Processing Unit (CPU), an Application Specific Integrated Circuit (ASIC), a Field Programmable Gate Array (FPGA), etc. The first control circuit 210 may be formed of, for example, a single integrated circuit, or may be formed of a plurality of integrated circuits in combination. The operation of the first control circuit 210 is executed in accordance with a program, etc., stored in a storage area within a memory circuit or a control circuit, for example.

For example, the first control circuit 210 obtains input signals to the output switch 121 of the surgical instrument 110, the touch screen 272, the input unit 274, and the microphone 276 of the first generator 200, etc. The first control circuit 210 controls display on the touch screen 272.

The first generator 200 includes a drive circuit 220. The drive circuit 220 is a circuit for driving the ultrasonic transducer 140. Namely, the drive circuit 220 is a circuit configured to generate a voltage to be applied to the electrodes of the ultrasonic transducer 140. The first generator 200 according to the present embodiment adopts a PLL control system as a resonance tracking system. The first generator 200 further adopts a constant current control system as an amplitude control system.

The drive circuit 220 includes a transformer 222. The transformer 222 boots a voltage, thereby outputting a necessary voltage. Outputs of the transformer 222 are input to the ultrasonic transducer 140. The transformer 222 has a further role of preventing a current containing a DC component from flowing between circuits of the first generator 200 and circuits of the surgical instrument 110. Herein, the circuits of the surgical instrument 110 are those which come into contact with a patient. The circuits of the first generator 200 are those which are connected to a commercial power source.

The drive circuit 220 includes a detection circuit 224, a power amplifier 226, a voltage control amplifier 228, a phase-locked loop (PLL) circuit 230, a differential amplifier 232, and a digital to analog converter (DAC) 234.

Outputs of the voltage control amplifier 228 are input to the power amplifier 226. The power amplifier 226 amplifies an output of the voltage control amplifier 228, thereby adjusting a signal to be input to the transformer 222. The detection circuit 224 detects a voltage and a current, which are input from the power amplifier 226 to a primary coil of the transformer 222, as well as phases of these voltage and current.

The PLL circuit 230 is a circuit for tracking a resonance frequency of the ultrasonic transducer 140. The PLL circuit 230 performs resonance tracking by using a voltage phase signal and a current phase signal which are detected by the detection circuit 224. The PLL circuit 230 performs resonance tracking by controlling a phase difference between a voltage and a current in such a manner that the phase difference approximates to zero. Outputs of the PLL circuit 230 are input to the voltage control amplifier 228. The voltage control amplifier 228 is a multiplier. In addition to signals from the PLL circuit 230, signals from the differential amplifier 232 are input to the voltage control amplifier 228. The differential amplifier 232 compares a signal of a current magnitude obtained from the detection circuit 224 with a signal from the DAC 234. The DAC 234 inputs to the differential amplifier 232 a signal relating to a magnitude of an output from the first control circuit 210 to the ultrasonic transducer 140.

When the output switch 121 of the surgical instrument 110 is turned on, the first control circuit 210 detects this fact. At this time, the first control circuit 210 outputs to the DAC 234 a digital signal relating to a target value of a magnitude of an output to the ultrasonic transducer 140. The DAC 234 converts a signal input from the first control circuit 210 into an analog signal, thereby outputting the converted signal to the differential amplifier 232.

The differential amplifier 232 receives inputs of signals from the DAC 234 and signals from the detection circuit 224. The differential amplifier 232 compares a signal from the DAC 234 with a signal from the detection circuit 224. In other word, the differential amplifier 232 compares a control signal indicative of a target value of an output from the first control circuit 210 with a present output detected by the detection circuit 224. The differential amplifier 232 outputs a comparison result to the voltage control amplifier 228.

The voltage control amplifier 228 multiplies an input from the PLL circuit 230 by an input from the differential amplifier 232. As a result, an output voltage is adjusted to have a magnitude indicated by the first control circuit 210. Thus, a signal on which resonance tracking has been performed by the PLL circuit 230, which corresponds to an output adjusted to have a magnitude based on a control signal of the first control circuit 210 using the voltage control amplifier 228 and the differential amplifier 232, is input to the power amplifier 226. The power amplifier 226 amplifies a signal, thereby outputting it to the transformer 222. The transformer 222 boosts an input voltage, thereby supplying a power to the ultrasonic transducer 140. In this manner, an energy is supplied as appropriate to the ultrasonic transducer 140. As a result, the ultrasonic transducer 140 vibrates.

The second generator 300 includes a second control circuit 310. The second control circuit 310 controls an operation of each element in the second generator 300. The second control circuit 310 is formed by an integrated circuit such as, e.g., a Central Processing Unit (CPU), an Application Specific Integrated Circuit (ASIC), a Field Programmable Gate Array (FPGA), etc. The second control circuit 310 maybe formed of, for example, a single integrated circuit, or may be formed of a plurality of integrated circuits in combination. The operation of the second control circuit 310 is executed in accordance with a program, etc., stored in a storage area within a memory circuit or a control circuit, for example.

For example, the second control circuit 310 obtains input signals to the output switch 121 of the surgical instrument 110, the touch screen 372 and the input unit 374 of the second generator 300, etc. The second control circuit 310 controls display on the touch screen 372. The first control circuit 210 of the first generator 200 and the second control circuit 310 of the second generator 300 are connected to each other. The first control circuit 210 of the first generator 200 and the second control circuit 310 of the second generator 300 exchange necessary information with each other.

The second generator 300 includes an output circuit 320. The output circuit 320 is a circuit configured to apply a high-frequency voltage between the probe 130 of the surgical instrument 110 and the return electrode 180. The output circuit 320 includes a power source circuit 322, a resonance circuit 324, a detection circuit 326, and a transformer 328.

For example, the power source circuit 322 is a DC/DC converter. The second control circuit 310 turns on an output of the power source circuit 322 based on the output switch 121 of the surgical instrument 110 being turned on. At this time, the second control circuit 310 controls a magnitude of an output of the power source circuit 322. A DC voltage output from the power source circuit 322 is supplied to the resonance circuit 324.

The resonance circuit 324 converts the DC voltage supplied from the power source circuit 322 into an AC voltage under control of the second control circuit 310. The resonance circuit 324 inputs the generated AC voltage to a primary coil of the transformer 328 via the detection circuit 326.

The detection circuit 326 detects, for example, a voltage applied to the primary coil of the transformer 328 and a current flowing through the primary coil. The detection circuit 326 transmits a detection result to the second control circuit 310. The second control circuit 310 controls an output of the power source circuit 322 using the detection result from the detection circuit 326.

The transformer 328 boosts an output voltage of the resonance circuit 324. The transformer 328 applies the boosted voltage between the probe 130 of the surgical instrument 110 and the return electrode 180. The transformer 328 has a further role of preventing a current containing a DC component from flowing between circuits of the second generator 300 and circuits of the surgical instrument 110. Herein, the circuits of the surgical instrument 110 are those which come into contact with a patient. The circuits of the second generator 300 are those which are connected to a commercial power source.

[Operation of Surgical System]

In treatment using the surgical instrument 110, a user brings the probe 130 into contact with a living tissue as a treatment target and turns on the output switch 121. At this time, the surgical instrument 110 outputs energy. For example, when the output switch 121 is turned on, the first control circuit 210 of the first generator 200 obtains information indicating that the output switch 121 has been turned on. The first control circuit 210 outputs to the drive circuit 220 a power for driving the ultrasonic transducer 140. With this output power, the ultrasonic transducer 140 vibrates. Upon receipt of transmission of this vibration, the probe 130 vibrates. At this time, the second control circuit 310 of the second generator 300 causes the output circuit 320 to output a high-frequency power. As a result, a high-frequency current flows through a living tissue interposed between the probe 130 and the return electrode 180.

In a portion in which a living tissue and the probe 130 are in contact, the living tissue generates heat by a high-frequency current flowing therethrough. Furthermore, vibration of the probe 130 prevents a cauterized living tissue from sticking to the probe 130. Meanwhile, cutting or hemostasis of a living tissue may also be realized with heat caused by friction between the living tissue and the probe 130 that vibrates at a frequency in an ultrasonic region.

The surgical system 10 according to the present embodiment uses a PLL in order to cause a vibration system including the ultrasonic transducer 140 and the probe 130 to resonate at a frequency in an ultrasonic region. Generally, a certain amount of time is required to cause resonance using a PLL. Therefore, in order to perform an output of a high-frequency power concurrently with vibration of the probe 130, such an output of a high-frequency power needs to be performed after resonance of the probe 130 is realized.

For this reason, the surgical system 10 according to the present embodiment is provided with two output modes. A first mode is a mode in which resonance of the probe 130 is realized in advance before the output switch 121 is pressed. A second mode is a mode in which resonance of the probe 130 is realized after the output switch 121 is pressed, and thereafter an output of a high-frequency power is performed.

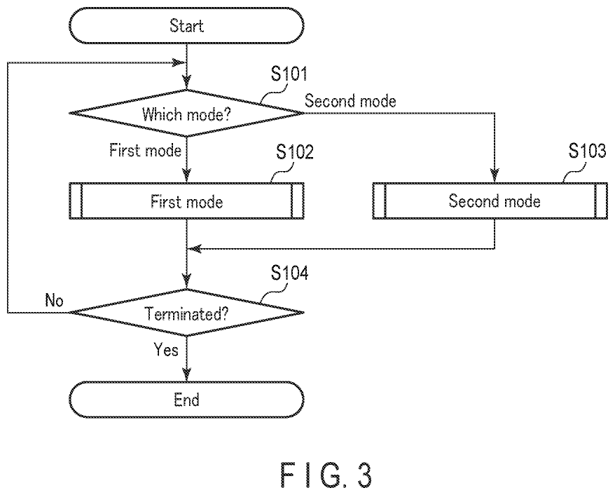

An output operation will be described with reference to a flowchart. As shown in FIG. 3, in step S101, for example, the first control circuit 210 of the first generator 200 determines which of the first mode and the second mode is currently set. An output mode is selected in advance by a user's input via the touch screen 272 of the first generator 200 before start of use. Herein, the description will be given for an example in which the first mode or the second mode is set by the first generator 200, and this setting is determined by the first control circuit 210 of the first generator 200. However, this example is not a limitation, and the setting and determination may be performed by the second generator 300.

In the case where the first mode has been set, the processing proceeds to step S102 to perform treatment in the first mode. In the case where the second mode has been set, the processing proceeds to step S103 to perform treatment in the second mode. After the operation in each mode, the processing proceeds to step S104.

In step S104, the first control circuit 210 of the first generator 200 determines whether to terminate the processing. For example, when the first generator 200 is powered off, it is determined that the processing is to be terminated. When the processing is not to be terminated, it returns to step S101. On the other hand, when the processing is to be terminated, it is terminated.

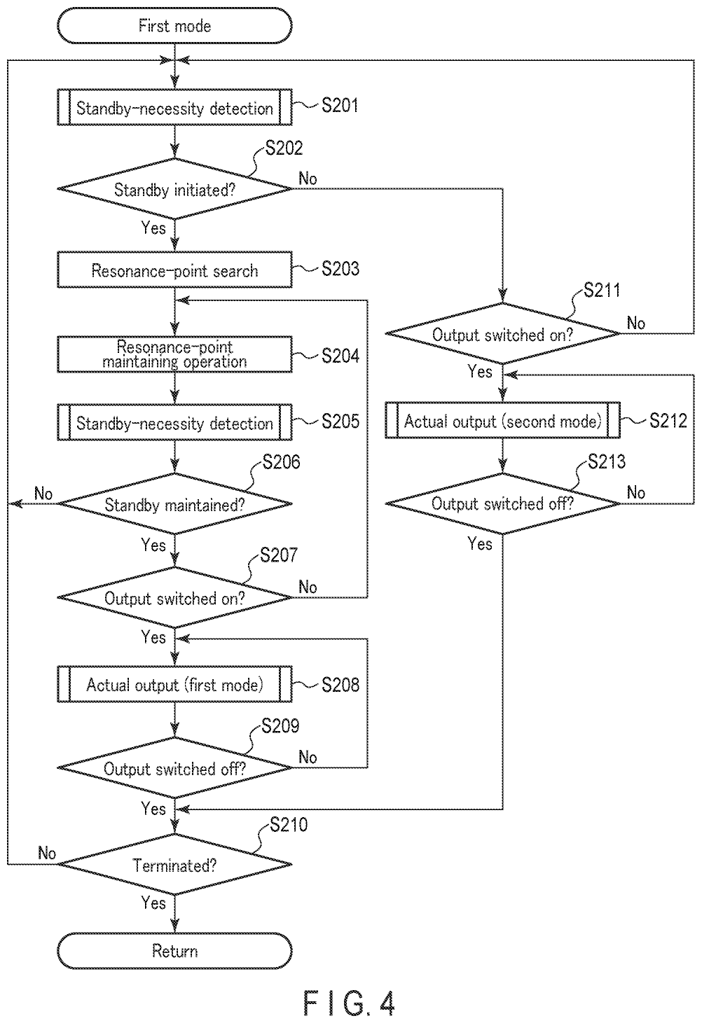

The processing in the first mode will be described with reference to the flowchart shown in FIG. 4. The first mode is a mode in which resonance of the probe 130 is realized in advance before the output switch 121 is pressed. When the output switch 121 is pressed, vibration of the probe 130 for treatment is initiated concurrently with an output of a high-frequency power.

In step S201, the first control circuit 210 of the first generator 200 performs standby-necessity detection processing for determining whether standby is necessary or not. Herein, standby means a state in which resonance of the probe 130 is maintained. When standby is necessary, a transition is made to a standby state. A criterion for determining the need for standby will be referred to as a first trigger.

For example, standby is determined to be necessary when the probe 130 is in contact with a living tissue. A determination as to whether the probe 130 and a living tissue are in contact is made by, for example, obtaining a value that reflects an electric impedance between the probe 130 and the return electrode 180. When the probe 130 and a living tissue are in contact with each other, an impedance between the probe 130 and the return electrode 180 connected to the living tissue is relatively low. On the other hand, when the probe 130 and a living tissue are separated from each other, an impedance between the probe 130 and the return electrode 180 is relatively high. Accordingly, the second generator 300 causes a feeble current to flow between the probe 130 and the return electrode 180, thereby obtaining a value relating to an impedance therebetween. The second control circuit 310 transmits this value to the first control circuit 210 of the first generator 200. The first control circuit 210 of the first generator 200 performs standby-necessity detection processing based on the received value. In this case, the first trigger is that an impedance between the probe 130 and the return electrode 180 falls below a predetermined value. In step S202, the first control circuit 210 determines based on a processing result in step S201 whether to initiate a standby state. To initiate a standby state, the processing proceeds to step S203. After step S203, a standby operation is performed. The standby operation includes searching for a resonance point by performing resonance-point scanning on the vibration system in response to the detection of the first trigger, and maintaining resonance of the vibration system if the resonance point is found.

In step S203, the first control circuit 210 causes the drive circuit 220 to perform a resonance-point search. In the resonance-point search, an output of the drive circuit 220 is adjusted to be low, and the probe 130 is caused to vibrate slightly by the ultrasonic transducer 140. The first control circuit 210 changes an output frequency gradually from a high frequency to a low frequency, for example. The first control circuit 210 analyzes a phase difference between a voltage and a current which are detected by the detection circuit 224 at the time when an output frequency is changed as described above. In a resonance point of the vibration system including the ultrasonic transducer 140 and the probe 130, a phase difference between a voltage and a current is eliminated. In a resonance point, a current easily flows. The first control circuit 210 performs a resonance-point scanning which determines a resonance point based on a phase between a voltage and a current, while changing an output frequency gradually from a high frequency to a low frequency, for example. In a resonance point, a current easily flows to lead to an increased amplitude. Thus, when a resonance point is found, the first control circuit 210 adjusts a current value to fall within an appropriate range so as to suppress an amplitude as appropriate. If a resonance point is found, the processing proceeds to step S204.

In step S204, the first control circuit 210 causes the drive circuit 220 to perform a resonance-point maintaining operation. In the resonance-point maintaining operation, the ultrasonic transducer 140 is kept vibrating with a low output and PLL processing is continuously performed. Since an output level is low, a vibration amplitude of the probe 130 is small, too. For this reason, even if the probe 130 comes into contact with a living tissue, the living tissue is not treated from this contact.

In step S205, the first control circuit 210 performs standby-necessity detection processing as in step S201.

In step S206, the first control circuit 210 determines whether to maintain a standby state. When a standby state is not to be maintained, an output to the ultrasonic transducer 140 is stopped and the processing returns to step S201. For example, when the probe 130 and a living tissue are separated from each other, it is determined that a standby state is not to be maintained. When the first trigger is canceled in this manner, power supply to the ultrasonic transducer 140 is stopped. On the other hand, if it is determined that a standby state is to be maintained, the processing proceeds to step S207.

In step S207, the first control circuit 210 determines whether the output switch 121 is on or not. If the output switch 121 is not on, the processing returns to step S204. Namely, resonance is maintained while a standby state is to be maintained. On the other hand, if it is determined that the output switch 121 is on, the processing proceeds to step S208.

In step S208, the surgical system 10 performs a main-output operation in the first mode. That is, the first control circuit 210 of the first generator 200 raises an output of the drive circuit 220 to a level necessary for the probe 130 to vibrate with an amplitude optimal for treatment. At this time also, resonance of the probe 130 is continuously maintained. As a result, the probe 130 vibrates with a sufficiently large amplitude. At the same time, the second control circuit 310 of the second generator 300 sets an output of the output circuit 320 to an output necessary for treatment with a high-frequency current. As a result, a high-frequency current flows from the probe 130 to a living tissue. With this high-frequency current and the vibration of the probe 130, the living tissue is treated. A criterion which leads a living tissue to be treated will be referred to as a second trigger. The output switch 121 being turned on is the second trigger. Similarly, the footswitch 190 being turned on is also the second trigger.

In step S209, the first control circuit 210 of the first generator 200 determines whether the output switch 121 is off or not. If an output remains in an on state without switching to off, the processing returns to step S208. Specifically, an actual output is maintained, the probe 130 vibrates with a large amplitude, and a high-frequency current flows from the probe 130 to a living tissue. Even if the probe 130 and a living tissue are separated from each other, the actual output is maintained while the output switch 121 remains on.

In step S209, if it is determined that the output switch 121 has been turned off, the processing proceeds to step S210. In step S210, the surgical system 10 stops an output. In step S210, the first control circuit 210 of the first generator 200 determines whether to terminate the processing in the first mode. If it is determined that the processing in the first mode is to be terminated, the processing is terminated. For example, when treatment is completed and the power is turned off, this processing is terminated. In addition, when a mode switching operation is performed via a menu screen so that the first mode is switched to the second mode, this processing is terminated. On the other hand, if it is determined that the processing is not to be terminated, the processing returns to step S201. Namely, the processing described above is repeated.

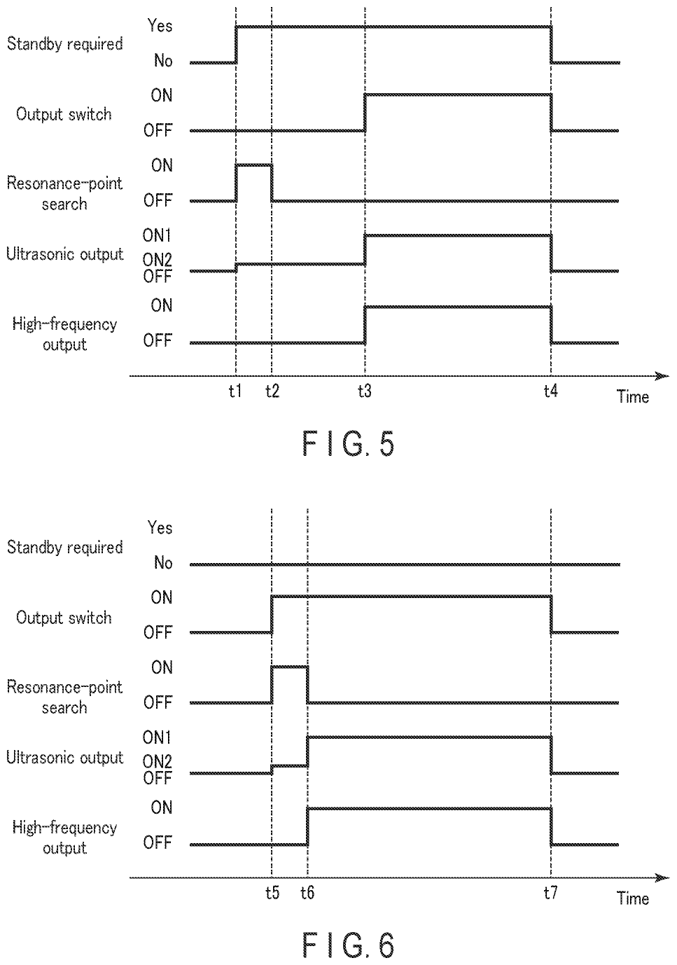

The operation in each element in the first mode will be described with reference to a timing chart shown in FIG. 5. Assume that standby becomes necessary at time t1. Herein, the output switch 121 still remains off. At this time, a resonance-point search is performed with a low output. Assume that a resonance point is found at time t2. Even after time t2, an output for driving the ultrasonic transducer 140 of the drive circuit 220 is maintained to be a low output for maintaining resonance.

Assume that the output switch 121 is turned on at time t3. At this time, an output for driving the ultrasonic transducer 140 of the drive circuit 220 is set to a high output. Furthermore, an output of the output circuit 320 is set to a high output, and an output of a high-frequency power is initiated. At time t4, if the output switch 121 is turned off, an output of the drive circuit 220 is stopped, so that vibration of the ultrasonic transducer 140 is stopped. Furthermore, an output of the output circuit 320 is stopped, so that an output of a high-frequency power is stopped.

The description will be continued by referring back to FIG. 4. In step S202, if it is determined that a standby state is not to be initiated, the processing proceeds to step S211. In step S211, the first control circuit 210 of the first generator 200 determines whether the output switch 121 is on or not. If the output switch 121 is not on, the processing returns to step S201. Namely, the processing described above is repeated. On the other hand, if it is determined that the output switch 121 is on, the processing proceeds to step S212.

In step S212, the surgical system 10 performs the actual output. At this time, a resonance-point search is not performed, so that the actual output is performed in the second mode. In step S213, the first control circuit 210 of the first generator 200 determines whether the output switch 121 is turned off or not. If the output switch 121 is not turned off and the on state is maintained, the processing returns to step S212 and the actual output is maintained. On the other hand, if the output switch 121 is turned off, the processing proceeds to step S210.

The operation in each element in the second mode will be described with reference to a timing chart shown in FIG. 6. Assume that the output switch 121 is turned on at time t5. At this time, a resonance-point search is performed with a low output. Assume that a resonance point is found at time t6. At this time, an output for driving the ultrasonic transducer 140 of the drive circuit 220 is set to a high output. Furthermore, an output of the output circuit 320 is set to a high output, and an output of a high-frequency power is initiated.

Switching to a high output of the drive circuit 220 and initiation of an output of the output circuit 320 may be performed based on a lock signal indicating that a resonance point has been found and resonance has been achieved. Specifically, the first control circuit 210 of the first generator 200 outputs such a lock signal to the second control circuit 310 of the second generator 300. The second control circuit 310 may initiate an output of a high-frequency power upon receipt of a lock signal. In addition, a period of time required for past resonance-point searches may be stored and used. Specifically, after this period of time has elapsed since the output switch 121 is turned on, switching to a high output of the drive circuit 220 and initiation of an output of the output circuit 320 may be performed.

At time t7, if the output switch 121 is turned off, an output of the drive circuit 220 is stopped, so that the vibration of the ultrasonic transducer 140 is stopped. Furthermore, an output of the output circuit 320 is stopped, so that an output of a high-frequency power is stopped.

As described above, in the first mode, the need for standby is determined. When a standby state is necessary for the reason that, for example, the probe 130 and a living tissue are brought into contact, a resonance-point search is performed, and resonance is maintained while the standby state is to be maintained. As a result, an ultrasonic output and a high-frequency output are concurrently initiated immediately after the output switch 121 is turned on. On the other hand, if the output switch 121 is turned on while a state is not a standby state, a resonance-point search is performed first and then an ultrasonic output and a high-frequency output are concurrently initiated as soon as a resonance point is found.

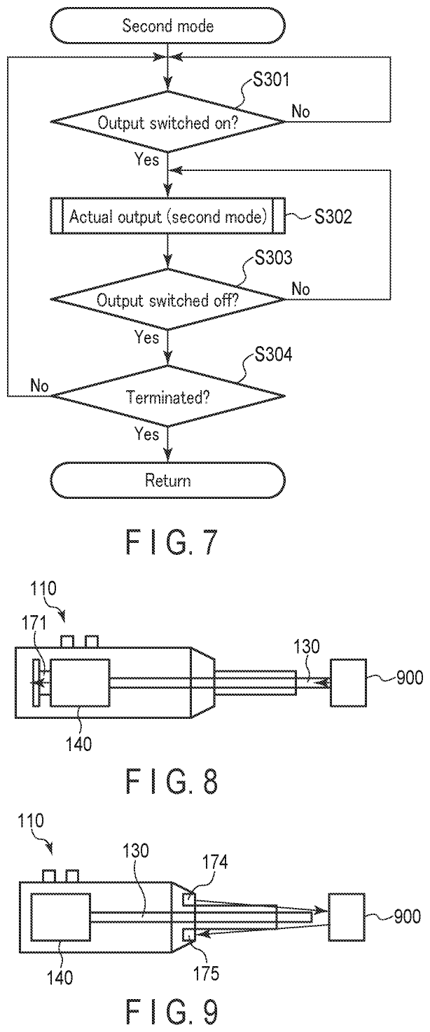

The operation in the second mode performed at step S103 will be described with reference to a flowchart shown in FIG. 7.

At step S301, the first control circuit 210 of the first generator 200 determines whether the output switch 121 is on or not. If the output switch 121 is not on, the processing returns to step S301. Namely, the processing waits until the output switch 121 is turned on. On the other hand, if it is determined that the output switch 121 is on, the processing proceeds to step S302.

At step S302, the surgical system 10 performs the actual output in the second mode described above. At step S303, the first control circuit 210 of the first generator 200 determines whether the output switch 121 is turned off or not. If the output switch 121 is not on and the on state is maintained, the processing returns to step S302 and the actual output is maintained. On the other hand, if the output switch 121 is turned off, the processing proceeds to step S304.

At step S304, the first control circuit 210 of the first generator 200 determines whether to terminate this processing. If it is determined that the processing is to be terminated, the processing is terminated. On the other hand, if it is determined that the processing is not to be terminated, the processing returns to step S301. Namely, the processing described above is repeated.

According to the second mode, no output is necessary until the output switch 121 is turned on. For example, an output for grasping a contact state between the probe 130 and a living tissue is not necessary.

A resonance-point search takes several hundred milliseconds, for example. If the vibration of the probe 130 is delayed with respect to an output of a high-frequency power, the effect of preventing a tissue from sticking to the probe 130 cannot be obtained while the high-frequency power is output alone. The surgical system 10 according to the present embodiment makes an adjustment in such a manner that the vibration at an ultrasonic frequency of the probe 130 and an output of a high-frequency power are initiated concurrently. In particular, in the first mode, a resonance-point search is performed in advance in such a manner that an output is performed immediately after a user turns on the output switch 121. Therefore, the surgical system 10 thus configured is highly functional and easy to use.

[Standby-Necessity Detection]

In the embodiment described above, the description was given to the example in which the need for a standby state for maintaining resonance is determined based on whether the probe 130 comes into contact with a living tissue or not. The description was further given to the example in which whether the probe 130 comes into contact with a living tissue or not is determined based on a value relating to an impedance between the probe 130 and the return electrode 180. However, these examples are not limitations. Specifically, various types of information may serve as the first trigger.

For example, a contact state between the probe 130 and the return electrode 180 may be determined based on a value of an output voltage or an output current instead of an impedance. As described above, a variation in various electrical parameters may serve as the first trigger.

For example, as shown in FIG. 8, the surgical instrument 110 may be provided with a pressure sensor 171 configured to detect that the probe 130 comes into contact with a living tissue 900. The pressure sensor 171 may be positioned at any location as long as the pressure sensor 171 can detect a contact pressure between the probe 130 and a living tissue. A contact pressure is detected by, for example, the probe 130 being pressed toward the proximal end. The first control circuit 210 of the first generator 200 obtains a detection result of the pressure sensor 171, thereby being able to determine whether or not the probe 130 is in contact with the living tissue 900. When the probe 130 is in contact with the living tissue 900, the first control circuit 210 is able to determine that a standby state is necessary. The pressure sensor is one example, and various dynamic sensors such as a strain sensor may be used in a similar manner.

Furthermore, an optical distance sensor capable of measuring a distance to an object which is present in the direction toward the distal end of the probe 130 may be used. For example, as shown in FIG. 9, the surgical instrument 110 may be provided with a light radiation unit 174 configured to emit, e.g., infrared rays in the direction toward the distal end of the probe 130, and a light reception unit 175 configured to detect light reflected by the living tissue 900, etc. The first control circuit 210 of the first generator 200 obtains a detection result by such an optical distance sensor, thereby being able to obtain a distance between the distal end of the probe 130 and an object such as the living tissue 900. The first control circuit 210 is able to determine that a standby state is necessary when a distance between the distal end of the probe 130 and an object falls below a predetermined value. The distal end of the probe 130 and an object are not necessarily in contact with each other.

A distance between the distal end of the probe 130 and an object may be specified based on a captured image of the distal end portion of the probe 130. For example, the first control circuit 210 captures an image of the distal end portion of the probe 130. The first control circuit 210 performs image processing on this image, thereby specifying a distance between the probe 130 and a living tissue. The first control circuit 210 may determine based on this specification result of a distance whether a standby state is necessary or not. As described above, the first control circuit 210 may monitor the first trigger based on image information.

Furthermore, a switch for switching to a standby state may be provided. For example, the output switch 121 may be a two-stage switch which is configured in such a manner that a standby state is initiated when a first-stage switch is turned on, and the actual output is performed when a second-stage switch is turned on. In this case, the presence/absence of standby may be set by a user using a user interface.

The first generator 200 maybe configured in such a manner that a user can vocally give his or her instruction to initiate a standby state. For example, a voice is collected by the microphone 276, and the first control circuit 210 performs voice recognition processing. Based on a result of voice recognition, the first control circuit 210 may determine whether a user desires a standby state or not. In this manner, the first control circuit 210 may monitor the first trigger based on voice information.

As described above, the need for a standby state is determined based on various types of information.

[Modifications]

In the embodiments described above, the description was given to the example in which an output level of the drive circuit 220 is smaller than an actual output. However, this example is nota limitation. At the time of resonance, an output level of the drive circuit 220 may always be equal to an actual output.

The roles between the first control circuit 210 of the first generator 200 and the second control circuit 310 of the second generator 300 in the surgical system 10 described in the above embodiments are only one example. Each of these control circuits may play any role. Furthermore, the first generator 200 and the second generator 300 may be integrated and a single control circuit may be provided.

In the embodiments described above, an output of the surgical instrument 110 corresponds to a combination of an output of a high-frequency power and vibration at a frequency in an ultrasonic region. However, an output of the surgical instrument 110 is not limited thereto. The surgical instrument may treat a living tissue using, for example, only vibration.

Furthermore, the surgical instrument maybe configured in such a manner that the distal end of the probe 130 is provided with a heater, for example. In such a case, the surgical instrument uses an output of heat from the heater and vibration at a frequency in an ultrasonic region. In this case also, the surgical instrument may be configured and function as in the embodiments described above.

Additional advantages and modifications will readily occur to those skilled in the art. Therefore, the embodiment in its broader aspects is not limited to the specific details and representative embodiments shown and described herein. Accordingly, various modifications may be made without departing from the spirit or scope of the general inventive concept as defined by the appended claims and their equivalents.

1. A generator comprising:
a drive circuit connected to a surgical instrument, the surgical instrument including a vibration system having a probe and an ultrasonic transducer, the drive circuit being configured to supply power to the ultrasonic transducer; and
at least one control circuit configured to:
detect a first trigger causing the at least one control circuit to perform a resonance-point search on the vibration system, the first trigger being based on the probe of the surgical instrument contacting a living tissue;
upon detecting the first trigger, determine a resonance point of a frequency of vibration of the probe by performing the resonance-point search on the vibration system;
upon determining the resonance point, cause the drive circuit to execute a standby operation by supplying power to the ultrasonic transducer so as to maintain resonance of the frequency based on the determined resonance point;
detect a second trigger causing the surgical instrument to treat the living tissue; and
upon detecting the second trigger, cause the drive circuit to execute an actual output operation by increasing the supply of power to the surgical instrument such that the surgical instrument treats the living tissue.

2. The generator according to claim 1, wherein during the standby operation, upon detecting cancelation of the first trigger before detection of the second trigger, the at least one control circuit causes the drive circuit to stop supplying the power that maintains resonance of the frequency of vibration of the probe.

3. The generator according to claim 1, wherein:
the at least one control circuit causes a power source to apply a voltage to the probe;
the generator further comprises a detection circuit configured to detect a variation in an electrical parameter that is varied by the probe contacting the living tissue; and
the at least one control circuit detects the first trigger based on an output of the detection circuit.

4. The generator according to claim 1, further comprising a dynamic sensor configured to detect a contact pressure between the probe and the living tissue, wherein the at least one control circuit detects the first trigger based on an output of the dynamic sensor.

5. The generator according to claim 1, further comprising a user interface to be operated by a user in order to generate the first trigger, wherein the at least one control circuit detects the first trigger based on an input to the user interface.

6. The generator according to claim 1, wherein the at least one control circuit is configured to:
obtain image information or voice information; and
detect the first trigger based on the obtained image information or the obtained voice information.

7. The generator according to claim 1, wherein the at least one control circuit increases power supplied to the ultrasonic transducer, as the actual output operation.

8. The generator according to claim 7, wherein:
the probe includes a high-frequency electrode for the surgical instrument to operate as a high-frequency treatment instrument;
the generator further comprises an output circuit configured to supply a high-frequency current to the high-frequency electrode; and
the at least one control circuit causes the output circuit to initiate supply of a high-frequency power to the high-frequency electrode as the actual output operation.

9. The generator according to claim 1, wherein:
the probe includes a high-frequency electrode for the surgical instrument to operate as a high-frequency treatment instrument;
the generator further comprises an output circuit configured to supply a high-frequency current to the high-frequency electrode; and
the at least one control circuit causes the output circuit to initiate supply of a high-frequency power to the high-frequency electrode, as the actual output operation.

10. The generator according to claim 1, wherein:
the at least one control circuit performs a first mode in which the standby operation is performed and then the actual output operation is performed, and a second mode in which the actual output operation is performed without performing the standby operation,
the generator further comprises a user interface to be operated by a user in order to switch between the first mode and the second mode, and
the at least one control circuit switches between the first mode and the second mode based on an input to the user interface.

11. An operation method of a generator, the method comprising:
supplying, by a drive circuit of the generator, a power to an ultrasonic transducer, the drive circuit being connected to a surgical instrument including a vibration system having a probe and the ultrasonic transducer;
detecting, by at least one control circuit, a first trigger that is generated in response to the probe contacting a living tissue, the first trigger causing the at least one control circuit to perform a resonance-point search on the vibration system;
upon detecting the first trigger, determining, by the at least one control circuit, a resonance point of a frequency of vibration of the probe by performing the resonance-point search on the vibration system;
upon determining the resonance point, causing, by the at least one control circuit, the drive circuit to execute a standby operation by supplying power to the ultrasonic transducer so as to maintain resonance of the frequency based on the determined resonance point;
detecting, by the at least one control circuit, a second trigger causing the surgical instrument to treat the living tissue; and
upon detecting the second trigger, causing, by the at least one control circuit, the drive circuit to execute an actual output operation by increasing the supply of power to the surgical instrument such that the surgical instrument treats the living tissue.
